(19) United States
(12) Patent Application Publication
(10) Pub. No.: US 2025/0239363 A1
(43) Pub. Date: Jul. 24, 2025

(54) METHOD FOR OPERATING A HEARING DEVICE, METHOD FOR OPERATING A HEARING DEVICE SYSTEM, AND HEARING DEVICE

(71) Applicant: Sivantos Pte. Ltd., Singapore (SG)

(72) Inventors: Rosa-Linde FISCHER, Erlangen (DE); Ronny HANNEMANN, Erlangen (DE); Nadja SCHINKEL-BIELEFELD, Erlangen (DE)

(21) Appl. No.: 19/034,855

(22) Filed: Jan. 23, 2025

(30) Foreign Application Priority Data
Jan. 23, 2024 (DE) 10 2024 200 600.9

(51) Int. Cl. G16H 40/67; A61B 5/11; H04R 25/00

(52) U.S. Cl. CPC G16H 40/67; A61B 5/112; A61B 2562/0219; H04R 25/604

(57) ABSTRACT

A method operates a hearing device that has sensor, in which the sensor is used to determine a current situation. The current situation is taken as a basis for checking whether a suitable time for an intervention for improving a state of health is at hand. Further, the method is used for operating a hearing device system and to a hearing device.

### CROSS-REFERENCE TO RELATED APPLICATION

This application claims the priority, under 35 U.S.C. § 119, of German Patent Application DE 10 2024 200 600.9, filed Jan. 23, 2024; the prior application is herewith incorporated by reference in its entirety.

### FIELD AND BACKGROUND OF THE INVENTION

The invention relates to a method for operating a hearing device, to a method for operating a hearing device system and to a hearing device. The hearing device contains a sensor.

Individuals who suffer from a reduction in their hearing normally use a hearing aid. This usually involves using an electromechanical sound transducer to capture ambient sound. The electrical signals generated on the basis of the ambient sound are amplified by means of an amplifier circuit and introduced into the auditory canal of the person by means of a further electromechanical transducer in the form of a receiver. The captured sound signals are usually also processed, which is normally accomplished using a signal processor of the amplifier circuit. This involves matching the gain to any hearing loss of the hearing aid wearer, who is subsequently also referred to as the beneficiary or wearer.

The gain or other signal processing components of the hearing aid is or are normally initially set by a specialist, for example an audiologist. It is then also usually possible for the beneficiary to change the settings during use/operation of the hearing aid and to customize them to their needs. This is usually accomplished by using the hearing aid to provide usually applicable training programs. This improves an, at least subjective, experience. This leads to greater acceptance of the hearing aid, and the beneficiary uses of the hearing aid to an increased extent.

If the beneficiary of the hearing aid has only a comparatively short period of time available, acceptance is usually (still) comparatively low, and the beneficiary does not use the hearing device very much. This is also sustained as a result of the settings not yet being fully customized to the needs of the wearer. The reduced acceptance and the lack of familiarization with the hearing device mean that some beneficiaries then do not carry out the training program. As a result, the hearing device is not fully customized to the needs of the beneficiary, and so acceptance is not increased further or may even decrease. It is also possible that the wearer has other illnesses in addition to the hearing loss that result in the beneficiary not carrying out/neglecting the training program.

To avoid the aforementioned problems, it is known practice to remind the beneficiary to carry out the training program. This is normally done in specific time periods, for example every 7 days. In this instance, it is possible that the beneficiary might want to carry out the training in principle, but that the time at which the reminder is provided is unsuitable, for example because the beneficiary is performing another task. When this task has ended, it is possible that the beneficiary will no longer remember the reminder, which is then not provided again until 7 days later. Beneficiaries usually switch off the reminder function if it was repeatedly provided at unsuitable times.

### SUMMARY OF THE INVENTION

The invention is based on the object of specifying a particularly suitable method for operating a hearing device and also a particularly suitable method for operating a hearing device system, and a particularly suitable hearing device, wherein in particular an acceptance by and/or a state of health of the beneficiary is increased.

The invention achieves this object in respect of the method for operating a hearing device by the features of the independent method for operating a hearing device claim in respect of the method for operating a hearing device system by the features of the independent method for operating a hearing device system and in respect of the hearing device by the features of the independent hearing device claim. Advantageous developments and configurations are the subject matter of the respective subclaims.

With the foregoing and other objects in view there is provided, in accordance with the invention, a method for operating a hearing device having a sensor. The method includes determining a current situation via the sensor, and taking the current situation as a basis for checking whether there is a suitable time for an intervention for improving a state of health.

The method is used for operating a hearing device. By way of example, the hearing device is an earphone or comprises an earphone, and the hearing device is, for example, a headset. Particularly preferably, however, the hearing device is a hearing aid. The hearing aid is used to assist a person suffering from a reduction in their hearing. In other words, the hearing aid is a medical device by means of which for example a partial hearing loss is compensated for. By way of example, the hearing aid is a receiver-in-the-canal (RIC) hearing aid, an in-the-ear hearing aid, an in-the-canal (ITC) hearing aid or a complete-in-canal (CIC) hearing aid, a pair of hearing aid spectacles or a pocket hearing aid. Alternatively, the hearing aid is a behind-the-ear hearing aid that is worn behind a pinna.

The hearing device is intended and configured to be worn on the human body. In other words, the hearing device preferably contains a retaining device by means of which it can be mounted on the human body. If the hearing device is a hearing aid, the hearing device is intended and configured to be arranged, for example, behind the ear or inside an auditory canal. In particular, the hearing device is wireless and intended and configured to be at least partially inserted into an auditory canal.

The hearing device preferably contains a microphone that is used to capture sound. In particular, operation results in the microphone being used to capture ambient sound, that is to say sound waves, or at least a portion thereof. The microphone is conveniently arranged at least partially inside a housing of the hearing device and therefore at least partially protected. The microphone is appropriately an electromechanical sound transducer. The microphone has, for example, just a single microphone unit or multiple microphone units that interact with one another. Each of the microphone units conveniently has a diaphragm that is made to vibrate on the basis of sound waves, the vibrations being converted into an electrical signal by means of an applicable recording device, such as a magnet that is moved in a coil. Alternatively, the microphone units are of capacitive design, and use is made of the circumstance that an applied voltage changes when the distance of the diaphragm from a static surface of the microphone unit changes. The voltage in this case is applied in particular between the diaphragm and the static surface. The microphone units are preferably of omnidirectional design. This, or another feature, means that it is at least possible to use the microphone to generate or at least provide an input signal that is based on the sound striking the microphone, specifically in particular the ambient sound.

The hearing device conveniently has a receiver for outputting an output signal. The output signal in this case is in particular an electrical signal and, for example, of digital or appropriately analog design. The receiver is preferably an electromechanical sound transducer, for example a loudspeaker. Depending on the design of the hearing device, in the intended state the receiver is arranged at least partially inside an auditory canal of a beneficiary of the hearing device, that is to say a person, who is also referred to as the wearer, beneficiary or hearing device wearer, or at least acoustically connected thereto. The hearing device is used in particular primarily to output the output signal by means of the receiver, with corresponding sound being produced. In other words, the main function of the hearing device is preferably to output the output signal.

The hearing device appropriately contains a signal processing unit by means of which the possible microphone and the possible receiver are connected for signal transfer purposes. The hearing device conveniently has a signal processor that, for example, forms the signal processing unit or at least is a component part thereof. The signal processor is, for example, a digital signal processor (DSP) or implemented by means of analog components. The signal processor or at least the signal processing unit is used in particular to customize the input signal generated by means of the microphone. The signal processing unit is at least suitable, in particular intended and configured, for this purpose. An A/D converter is conveniently arranged between the microphone and the signal processing unit, for example the signal processor, if the signal processor is in the form of a digital signal processor. Particularly preferably, the hearing device additionally comprises an amplifier, or at least part of the amplifier is formed by means of the signal processing unit. By way of example, the amplifier is connected upstream or downstream of the signal processor for signal transfer purposes.

The hearing device contains a sensor. The sensor can be used in particular to capture specific values and/or produce measurement data. These correspond, by way of example, to external influences or are at least influenced from outside the hearing device. In other words, the values are not or not only operating data/operating values of the hearing device itself. In other words, it is in particular possible to influence the values from outside the hearing device and/or independently thereof. The sensor is suitable, in particular intended and configured, for this purpose.

The method provides for the sensor to be used to determine a current situation. In other words, the sensor is conveniently used to capture, for example measure, the values, and these are used in particular to derive the current situation. The current situation conveniently describes a state that the hearing device is in and that is realized in particular on the basis of external specifications/circumstances. In other words, the current situation conveniently does not describe or does not only describe the operation of the hearing device and is preferably independent of the operation of the hearing device itself. In particular, the current situation at least partially describes the situation that the beneficiary using the hearing device is in. In particular, the current situation used is an ambient situation.

The current situation is taken as a basis for checking whether a suitable time for an intervention for improving a state of health is at hand. The intervention is used in this case to improve the state of health and is suitable, in particular intended and configured, for this purpose. The state of health is associated with the beneficiary of the hearing device and describes, by way of example, only the physical and/or mental condition of the beneficiary, preferably in the context of the hearing device. Improvement is accomplished in this instance for example by reducing a complaint in the beneficiary, or said beneficiary is prevented from experiencing an affliction or the affliction is prevented from increasing, in particular provided that the beneficiary pursues the intervention. The improvement is made in the short or long term, for example. The state of health relates, for example, to the emotional and/or physical state of the beneficiary. In particular, the improvement in the state of health results in an improvement in the lifestyle and/or productivity of the beneficiary.

The intervention comprises in particular the output of information, preferably an instruction for a task that the beneficiary can or is supposed to perform. Provided that the user complies with the information, that is to say preferably adapts their behavior accordingly, the improvement in the state of health is made. For example, the intervention/information is firmly predefined and/or independent of the current situation. By way of example, such an intervention is that the beneficiary is supposed to carry out a training program so that use of the hearing device is improved. Alternatively, the intervention contains the instruction to do some sport or, for example, to stand up, that is to say in particular to interrupt a lengthy period of sitting. Alternatively or in combination with this, the information is customized for the current situation. For example, the information then contains the prompt to leave a loud environment.

Preferably, if the time is suitable, the intervention is carried out, that is to say in particular an applicable output is made. This is accomplished, for example, by outputting an applicable audio output via the receiver. In addition to the information, a further signal is conveniently also output in this instance, for example background noise or background music, so that the beneficiary's attention is directed to the information. Alternatively, the receiver is used to output additional sound that masks existing background noise, that is to say in particular destructively interferes therewith. This also steers the beneficiary's attention to the information. By way of example, carrying out the intervention results in the hearing device being put into a different operating mode, in which in particular the opportunity to improve the state of health is provided for the beneficiary. In particular, the change of operating mode results in a gain factor being adjusted and/or a noise cancellation being changed.

The method therefore results in the time for the intervention not being determined statically or on the basis of external specifications. Rather, the sensor is used to determine the current situation and a check is then performed to ascertain whether the suitable time for intervention is at hand. The sensor can be used to determine the current situation comparatively accurately in this instance, at least from the subjective point of view of the beneficiary. It is therefore more likely that the beneficiary will not find the intervention disruptive and will comply with the information contained therein to improve the state of health. The increased likelihood therefore means that the state of health of the beneficiary is increased. Since the intervention is not carried out at unfavorable times, the beneficiary does not feel disrupted by it and so acceptance is increased. It is also unlikely that the beneficiary will prevent the output of such interventions, that is to say in particular switch off this functionality of the hearing device.

Particularly preferably, the sensor used is the microphone. This means that no additional hardware is required, in particular if the hearing device is in the form of a hearing aid or contains a noise cancelling unit. The microphone is therefore used to capture ambient sound, and the ambient sound is taken as a basis for determining the current situation. This makes it possible in particular to determine whether the beneficiary is in company, and/or whether they are in the open air or inside a building. It also makes it possible to check, for example, whether the beneficiary themselves is currently speaking. In this instance, it is in particular not a suitable time if the beneficiary themselves is speaking, that is to say if the microphone is used to capture the voice of the beneficiary themselves. If the beneficiary is otherwise engaged, listening to other persons and/or otherwise concentrating, the time is not suitable either, since the intervention for improving the state of health is then perceived as disruptive. By contrast, the time is suitable at least when no speech by the beneficiary and/or other persons themselves is captured.

Alternatively or in combination with this, the sensor used is an acceleration sensor. This is conveniently arranged inside a housing of the hearing device. The acceleration sensor can be used in particular to determine a current attitude/orientation of the hearing device and, from this, for example whether the beneficiary is sitting or standing. If the beneficiary has already been sitting for a comparatively long period of time, or at least has been sitting for longer than a specific predefined length of time, the intervention that is output is in particular the information that the beneficiary is supposed to get up. Alternatively or in combination with this, the acceleration sensor is taken as a basis for determining a gait of the beneficiary and using this to derive in particular the current situation, that is to say whether the beneficiary is running, standing and/or has a comparatively unsteady gait, for example. This is therefore also used in particular to determine whether the time is suitable for output for the intervention. Alternatively or in combination with this, the acceleration sensor is used to infer the current activity of the beneficiary, for example whether they are performing physical activity, and if necessary what physical activity they are performing. If the beneficiary is already doing sport, for example, the time is not suitable, since an improvement in the state of health is already being made.

Alternatively, the sensor contains an EEG electrode, for example. The electrode is used in particular to record an activity of an auricle and/or a brain activity of the beneficiary. It is therefore possible in particular to determine whether the beneficiary is currently concentrating on specific sounds or whether they are relaxed. If the beneficiary is concentrating, then, in particular, the time is not suitable for the intervention. By way of example, only one applicable sensor is present, or preferably multiple such sensors, which are in particular of different design. An accuracy for determining the current situation is therefore improved. By way of example, one of the sensors, preferably the microphone, is initially used to limit the possibilities for the current situation in this instance. Depending on the applicable limitation, another of the sensors, in particular the acceleration sensor, is used, for example, to limit the possibilities further, and only subsequently is the EEG electrode used, if present. The acceleration sensor/the EEG electrode is therefore used for determining the current situation only comparatively rarely, or at least only when the microphone has already been taken as a basis for determining indications of the existence of a current situation whose conclusive determination is made possible only on the basis of the acceleration sensor or the EEG electrode. Because of this, an energy requirement of the hearing device is reduced, the current situation also still being able to be determined comparatively accurately.

Particularly preferably, it is assumed that the suitable time is at hand when there is the opportunity for an activity that changes the state of health. In other words, the current situation reveals that there is the opportunity to perform the activity. If, for example, the current situation is taken as a basis for determining that there is the opportunity to carry out sports training, for example as part of an infrastructural specification, then the time is suitable. By way of example, this is accomplished by using the possible acceleration sensor to determine a step length, that is to say in particular whether the beneficiary is currently standing, walking or running. If the beneficiary is running, for example, the intervention that is output is the intervention that said beneficiary is supposed to continue to run for a further distance. The intervention therefore comprises a motivation. Alternatively, for example a comparatively high level of ambient sound results in the intervention that is output being the recommendation to look for a different environment. Alternatively or in combination with this, the opportunity for improvement that is used, for example, is the prompt to use the hearing device and/or to extend the period of use. In this instance, the time is assumed to be suitable if the beneficiary wishes to switch off the hearing device, or removes the hearing device.

Alternatively or in combination with this, it is assumed that the suitable time is at hand when no other persons are present. In other words, for the current situation, it is determined whether the other persons are present, that is to say in the environment of the wearer. For example, the interventions used in this case are the prompt to carry out a training program of the hearing device when no persons are present. Otherwise, the intervention is not carried out, as in this case it is already substantially certain that the beneficiary does not use the training program in the presence of other persons and will therefore not pursue the intervention. Rather, the beneficiary would feel disrupted on account of the intervention, or would not be able to follow a conversation, for example. The microphone is conveniently used to determine whether other persons are present. Alternatively or in combination with this, it is assumed that the time is not suitable if the beneficiary's own voice is captured.

Alternatively or in combination with this, it is assumed that a suitable time is at hand when a current reproduction setting does not correspond to the current situation. In other words, the intervention is carried out in particular if it would be possible to use an improved reproduction setting for the current situation, and so in particular an intelligibility is increased for the beneficiary. In particular, this is done if there are multiple speaking persons and/or if there is a comparatively low signal-to-noise ratio (SNR) in the output sound provided by means of the hearing device. The current reproduction setting does not correspond to the current situation, for example, because the current situation has changed and the current reproduction setting has not been customized for the current situation. In each of these cases, the intervention is dependent on the current situation. Alternatively, the intervention is independent of the current situation. The intervention is in particular not a changing of the reproduction setting, but rather, for example, the reminder to carry out the training program.

The method is used for operating a hearing device system that contains a hearing device. By way of example, the hearing device is an earphone or comprises an earphone. Particularly preferably, however, the hearing device is a hearing aid and the hearing device system is therefore a hearing aid system, which is also referred to as a hearing aid system. The hearing aid is used to assist a person suffering from a reduction in their hearing. In other words, the hearing aid is a medical device by means of which for example a partial hearing loss is compensated for. By way of example, the hearing aid is a receiver-in-the-canal (RIC) hearing aid, an in-the-ear hearing aid, an in-the-canal (ITC) hearing aid or a complete-in-canal (CIC) hearing aid, a pair of hearing aid spectacles, a pocket hearing aid, a bone conduction hearing aid or an implant. Alternatively, the hearing aid is a behind-the-ear hearing aid that is worn behind a pinna.

The hearing device contains a sensor and is operated using a method in which the sensor is used to determine a current situation. The current situation is taken as a basis for checking whether a suitable time for an intervention for improving a state of health is at hand. The hearing device conveniently contains a control unit that is suitable, in particular intended and configured, for carrying out at least part of the method.

The hearing device system further comprises a portable further device. The further device is in particular able to be connected to the hearing device for signal transfer purposes and is preferably connected to the hearing device for signal transfer purposes during the method. To this end, the hearing device and the further device conveniently each have a communication device. The signal transfer connection is made for example by wire or preferably wirelessly. In particular, the signal transfer connection meets a specific standard, for example a WLAN or Bluetooth standard. The further device is appropriately a smartphone or a wearable. In an alternative, the further device is, by way of example, another hearing device, which, for example, is similar to the hearing device or differs therefrom.

The method results in the further device being used to provide a parameter that is also taken as a basis for determining the current situation. The further situation is therefore determined both on the basis of the parameter and by means of the sensor. Due to the parameter, an accuracy for determining the current situation is improved.

By way of example, the intervention is carried out just by means of the hearing device or just by means of the further device. Carrying out the intervention results in the further device being put into a different operating mode, for example, in which the further device has a different interface, in particular if the further device is a smartphone or other wearable. The intervention is conveniently carried out by means of the further device when the further device is currently being used by the beneficiary. That is to say that if the portable further device is the smartphone, the intervention is output via a display and/or audibly, for example. If, on the other hand, the beneficiary is currently not using the further device directly, for example when the beneficiary is controlling a motor vehicle, the hearing device is used to carry out the intervention, that is to say in particular to audibly output the information. By way of example, the information is first output via the hearing device or the further device, and if the beneficiary does not react thereto, the information is output on the respective other device. The likelihood of the beneficiary perceiving the intervention is therefore increased. In particular, an estimated signal-to-noise ratio for the respective device is taken into account when selecting the respective device for outputting the information.

By way of example, the parameter used is a calendar entry. The calendar entry is in particular stored in the further portable device. If no calendar entry exists, the time is suitable, in particular. By way of example, it is necessary for there to be no calendar entry in order for the suitable time to be at hand. Alternatively or in combination with this, it is necessary for a calendar entry to have ended. The beneficiary therefore has sufficient time for improving the state of health. Alternatively, the time is suitable when the calendar entry that is used to specify that the improvement in the state of health is supposed to be made, that is to say in particular that a specific activity is carried out, exists. In this case, the time is suitable, at least provided that the sensor is not used to determine that the time is not suitable, for example because other persons are present in the environment.

Provided that the beneficiary has actually made the improvement to the state of health, for example has completed the training program, a new calendar entry is preferably created that is used as a parameter when the method is carried out subsequently. In this instance, a similar time to the time at which the improvement was made is used for the new calendar entry. The new time is moved by a specific period of time, for example one day, one week or one month. Alternatively or in combination with this, a time for which it is assumed that the beneficiary will be at the same location again and/or in a similar current situation is chosen for the new calendar entry. The likelihood of the beneficiary then also pursuing the intervention is therefore increased.

Alternatively or in combination with this, the parameter used, for example, is a current position. In this instance, in particular the further device has an opportunity to determine the current position, for example a GPS sensor. By way of example, the current situation is compared with stored map material in this instance. In particular, the time is suitable if, on the basis of the current position, it is determined that there are training opportunities close to the beneficiary, for example a keep-fit path or training equipment.

In one development, the calendar entry is input by the beneficiary. In this instance, in particular a time series is input by the beneficiary, that is to say multiple calendar entries that are at specific intervals of time from one another. The beneficiary therefore preferably indicates that they want to carry out a training program (hearing training), and can for this purpose stipulate a recurring time for carrying out the training program. In particular, the beneficiary also indicates which of multiple activities for improving the state of health (lifestyle) they want to perform. The method conveniently results in an initial likelihood of the suitable time being determined on the basis of the user input. This preferably also involves taking account of formulae or other inputs/factors that are used to specify when human beings normally carry out such hearing training.

The likelihood depends on multiple factors, determination involving information being used by multiple devices, at least by the hearing device and the further device. The information (parameter) used by the further device is, for example, a time of day, the current position (location), the preceding activity (e.g. movement, applications/actions used on the telephone) and/or the time since the last hearing training, this being determined on the basis of the applicable parameter used, specifically the respective calendar entry used. In particular multiple such parameters are used in this instance. It is also possible for there to be just one or more such further devices, one being a smartwatch, for example, the parameter provided being in particular a heart rate of the beneficiary. The information provided by means of the sensor, specifically for example the attitude/acceleration of the hearing device, the existence of own voice activity, the acoustic class and/or an acoustic level, is used for determining the likelihood.

If the beneficiary now carries out training/performs the activity to improve the state of health, it is assumed that the current situation is suitable therefor. The time of day then prevailing, the then current position/location, the acoustic environment then prevailing (e.g. own voice, speech) and/or activities immediately before the training/the performance of the activity (e.g. sitting down, operating the further device) is/are therefore stored. On the basis of these data, the function for determining the likelihood is conveniently updated.

If, when the function is performed at a later time, the determined likelihood exceeds a specific threshold value, the time is suitable, and the intervention is in particular carried out. The intervention is conveniently used to ask the beneficiary to carry out the hearing training. If the beneficiary does not want to carry out the intervention, it is in particular possible to indicate the reason for this (e.g.: “too stressed”, “too tired”, “the location is not good”, “other people are present”, “I have just carried out the training—I need a break”, “no further interest in hearing training”, and so on). On the basis of these inputs, the function for determining the likelihood is conveniently updated (e.g. “the location is not good” leads to a lower likelihood of this position/location and/or similar locations (e.g. if a park is not a good location, the likelihood of all locations that are loudly classified as a park in the possible map material is lowered). If the beneficiary does not react to the intervention and also does not indicate why the activity is not carried out, the intervention is carried out otherwise, for example audibly if the intervention was first carried out visually.

By way of example, it is assumed that the suitable time is at hand when the hearing device is not connected to the further device for signal transfer purposes. This is the case for example when the devices are at a comparatively great distance apart. This occurs for example when the beneficiary leaves the house with the further device but leaves the hearing device behind. In this case, the intervention that is output is in particular that the hearing device is supposed to be taken along. The parameter used in this instance is in particular the state of the signal transfer connection from the further device to the hearing device.

Alternatively or in combination with this, it is assumed that the suitable time is at hand when the hearing device is not worn on the body of the beneficiary. In this case, the hearing device is not currently being used to improve speech intelligibility for the beneficiary, and so the state of health of the beneficiary is impaired. Owing to the interventions being carried out, that is to say in particular the prompt that the hearing device is supposed to be used, the state of health of the beneficiary, in particular in conjunction with the hearing device, is therefore improved. In this instance, the intervention is conveniently carried out by means of the further device, the sensor of the hearing device being in particular taken as a basis for determining that the hearing device is not on the body.

In particular, the calendar entry is taken as a basis for checking whether a social and/or cultural activity is pending. In this case, the intervention is carried out in particular when the beneficiary is not using the hearing device, or the hearing device is not connected to the further device. This ensures that the beneficiary uses the hearing device for the social/cultural activity and can follow the conversations there comparatively well. This improves the state of health, at least for this period, since a limitation for the beneficiary is reduced.

The hearing device is, for example, a headset or particular preferably a hearing aid. For example, the hearing aid is a receiver-in-the-canal (RIC) hearing aid, an in-the-ear hearing aid, an in-the-canal (ITC) hearing aid or a complete-in-canal (CIC) hearing aid, a pair of hearing aid spectacles or a pocket hearing aid. Alternatively, the hearing aid is a behind-the-ear hearing aid that is worn behind a pinna.

The hearing device comprises a sensor and is operated using a method in which the sensor is used to determine a current situation. The current situation is taken as a basis for checking whether a suitable time for an intervention for improving a state of health is at hand. The hearing device conveniently contains a control unit that is suitable, in particular intended and configured, for carrying out at least part of the method.

The invention further relates to a hearing device system that comprises such a hearing device and also a portable further device, and that is operated using the method for operating a hearing device system described above. In particular, the further portable device and/or the hearing device therefore comprises an applicable control unit that is used to carry out at least part of the method. In other words, the control unit or the control units is or are therefore suitable, in particular intended and configured, for carrying out the method.

The developments and advantages explained in connection with the two methods also apply, mutatis mutandis, to the hearing device/the hearing device system and among one another, and vice versa.

Other features which are considered as characteristic for the invention are set forth in the appended claims.

Although the invention is illustrated and described herein as embodied in a method for operating a hearing device, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is an illustration showing a system containing a hearing device system having a hearing device and a further device in schematically simplified form, and

FIG. 2 is a flowchart showing a method for operating the hearing device system.

Mutually corresponding parts are provided with the same reference signs throughout the figures.

### DETAILED DESCRIPTION OF THE INVENTION

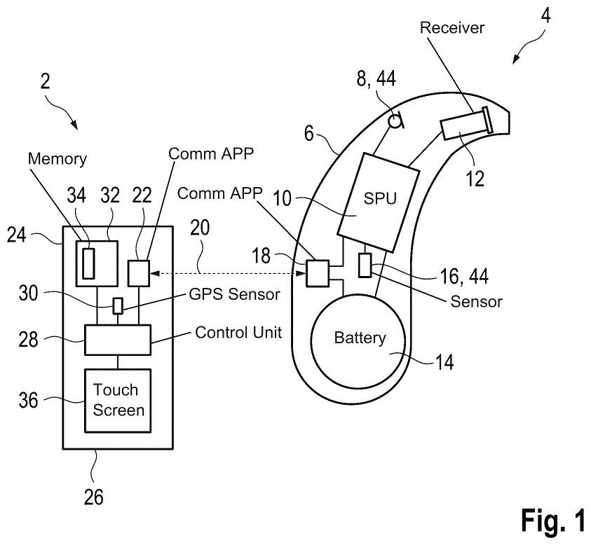

Referring now to the figures of the drawings in detail and first, particularly to FIG. 1 thereof, there is shown a hearing device system 2 having a hearing device 4 in the form of a hearing aid. It is therefore a hearing aid system. The hearing device 4 is intended and configured to be worn behind an ear of a beneficiary (user, hearing device wearer, wearer). In other words, it is a behind-the-ear hearing aid. The hearing device 4 has a (hearing device) housing 6, which is made from a plastic using an injection molding method. The hearing device housing 6 contains a microphone 8. The microphone 8 is coupled to electronics 10 for signal transfer purposes, said electronics containing a signal processing unit (not shown in more detail) having an amplifier circuit and a signal processor. The electronics 10 are formed by means of circuit elements, for example electrical and/or electronic components. The signal processor is a digital signal processor (DSP) and connected to the microphone 8 via an A/D converter (not shown in more detail) for signal transfer purposes.

The electronics 10, which act as a control unit of the hearing device 4, have a receiver 12 coupled to them for signal transfer purposes. During operation, the receiver 12 is used to convert an (electrical) signal provided by means of the electronics 10 into output sound, that is to say into sound (waves). These are introduced into a sound tube (not shown in more detail), one end of which is attached to the hearing device housing 6. The other end of the sound tube is enclosed by means of a dome, which in the intended state is arranged in an auditory canal of the beneficiary. The electronics 10, the microphone 8 and the receiver 12 are supplied with power by means of an energy store in the form of a battery 14, which is arranged in the hearing device housing 6.

The hearing device housing 6 further contains an acceleration sensor 16 by means of which an acceleration and a movement of the hearing device housing 6 can be detected. The acceleration sensor 16 is connected to the electronics 10, and the electronics 10 are used to evaluate the data/signals provided by the acceleration sensor 16. The hearing device housing 6 further has a communication apparatus 18 arranged inside it, which is also supplied with power by means of the battery 14.

The communication apparatus 18 can be used to make a signal transfer connection 20, specifically a radio connection, to a further communication apparatus 22, wherein the signal transfer connection 20 uses a Bluetooth standard. The two communication apparatuses 18, 22 are therefore of corresponding design. The further communication apparatus 22 is part of a portable further device 24 of the hearing device system 2 and arranged in a further housing 26 of the further device 24. The further device 24 is in the form of a smartphone. The further device 24 is associated with this hearing device 4, but in principle it is also possible for the further device 24 to be associated with other hearing devices 4.

The further housing 26 has a control unit 28 arranged inside it, which is connected to the further radio apparatus 22 for signal transfer purposes. The control unit 28 is further connected, for signal transfer purposes, to a GPS sensor 30 arranged inside the further housing 26 or to at least one sensor by means of which the position of the further housing 26 can be determined, in particular on a satellite -assisted basis.

Further, the control unit 28 is connected to a memory 32 of the further device 24 for signal transfer purposes, which memory stores a calendar entry 34. Moreover, the further device 24 comprises a touchscreen 36, which is used as an output and input device, and is likewise connected to the control unit 28 for signal transfer purposes.

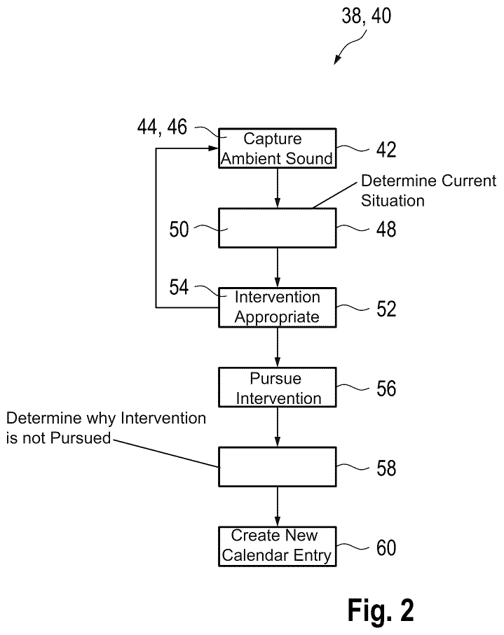

FIG. 2 shows a method 38 for operating the hearing device system 2, part of which is carried out by means of the control unit 28 and the electronics 10. The electronics 10 are used to carry out a method 40 for operating the hearing device 4. In other words, the method 38 for operating the hearing device system 2 includes the method 40 for operating the hearing device 4.

In a first work step 44, the microphone 8 is used to capture ambient sound, and the acceleration sensor 16 is used to detect a current orientation of the housing 6. The measurement data captured by means of the microphone 8 and the acceleration sensor 16 correspond to a situation that the hearing device 4 and therefore also the beneficiary of the hearing device system 2 are in. The microphone 8 and the acceleration sensor 16 are therefore each used as a sensor 44. Moreover, the control unit 28 is used to read a calendar entry 34 from the memory 32, or possibly multiple such calendar entries if they are available. Each calendar entry 34 is a parameter 46.

By way of example, the calendar entry 34 stores that the beneficiary wants to perform an activity or other task for improving a state of health of the beneficiary. By way of example, such an activity is carrying out a training program so that the beneficiary becomes accustomed to the hearing device 4 and/or a setting of the electronics 10 is changed, so that the electronics are customized comparatively accurately to the needs of the beneficiary. As an alternative, a social or cultural activity, for example, is stored in one of the calendar entries 34.

Moreover, the GPS sensor 30 is used to determine the current position of the further device 24. This is also one of the parameters 46.

In a subsequent second work step 48, a current situation 50 is determined. The current situation 50 is determined on the basis of the measurement data captured by means of the sensors 44 and on the basis of the parameters 46. The current situation 50 is determined by means of either the electronics 10 or the control unit 28, for which purpose the hearing device 4 transmits the measurement data determined by means of the sensors 44 to the further device 34, or the parameter(s) 46 from the further device 24 to the hearing device 4. In summary, the sensors 44 are therefore used, and the parameters 46 are taken as a basis, for determining the current situation 50, the parameters 46 being provided by means of the further device 24.

In a subsequent third work step 52, the current situation 50 is taken as a basis for checking whether a suitable time 54 for an intervention for improving the state of health is at hand. The suitable time 54 is not at hand in this case if the beneficiary would be disrupted by the intervention, for example because, on the basis of one of the calendar entries 34, it is specified that the beneficiary is in a meeting, which is confirmed on the basis of the measurement data captured by means of the sensors 44. The suitable time 54 is also not at hand if the beneficiary is currently taking part in a social or cultural activity, which is specified on the basis of one of the calendar entries 34 and confirmed by means of the sensors 44. The suitable time 54 is not at hand if other persons are present. In other words, it is assumed that the suitable time 54 is at hand only when no other persons are present.

If the suitable time 54 is not at hand, the first and second work steps 42, 48 are performed again subsequently, that is to say at a later time, and therefore the current situation 50 is determined again. This is done continuously, for example, or only in specific discrete time periods, for example every minute or every hour.

By contrast, the suitable time 54 is at hand when there is the opportunity for an activity that changes the state of health, that is to say for example if, on the basis of the GPS sensor 30 and a comparison with map material stored in the memory 32, it is evident that the beneficiary is at a training ground at which there is training equipment, for example. In summary, it is assumed that the suitable time 54 is at hand when there is the opportunity for the activity that changes the state of health. In this case, the activity that changes the state of health is physical exercise. However, it is also specified that one of the activities that changes the state of health is carrying out the training program of the hearing device 4. In this case too, the suitable time 54 is at hand only when no other persons are present, however.

It is also assumed that the suitable time 54 is at hand when the hearing device 4 is not worn on the body of the beneficiary, that is to say when the beneficiary is not using the hearing device 4, for example. The sensors 44 are used to determine the non-use in this case. One of the calendar entries 34 is used to specify that a social or cultural activity is subsequently taking place for which it is important that the beneficiary detects the environment correctly. The current situation 50 obtained is therefore that the hearing device 4 is not worn, and that the social or cultural activity is pending. Alternatively or in combination with this, it is assumed that the suitable time 54 is at hand when the hearing device 4 is not connected to the further device 24 for signal transfer purposes, that is to say when the signal transfer connection 20 is interrupted. This is the case, for example, when the hearing device 4 and the further device 40 are at a comparatively great distance from one another. In another alternative, it is assumed that the suitable time 54 is at hand when a current reproduction setting does not correspond to the current situation 50.

If the suitable time 54 is at hand, the intervention is carried out in a subsequent fourth work step 54. This is accomplished by outputting applicable information, for example by means of the receiver 12 or via the touchscreen 36, that is to say visually. In other words, the intervention is carried out by means of the hearing device 4 or the further device 24, for example.

If there is the opportunity for the activity, the intervention that is output is the information that the beneficiary is supposed to carry out said activity. If the current reproduction setting does not correspond to the current situation 50, the beneficiary is made aware on the basis of the intervention that they are supposed to carry out the training program of the hearing device 4, for example, so that they can appropriately customize the reproduction input setting for the current situation 50. In this instance, the intervention is appropriately first carried out by means of the hearing device 4, in particular if the beneficiary is moving. If, on the other hand, the beneficiary is using the further device 24, the intervention is carried out by means of the further device 44. If the beneficiary is not wearing the hearing device 4, the intervention is in particular carried out by means of the further device 24. If the beneficiary initially does not react to the intervention, the intervention is subsequently carried out using the respective other device 4, 24, with the result that the information is reliably conveyed to the beneficiary.

If the beneficiary carries out the intervention, the values characterizing the current situation 50, that is to say in particular the measurement data captured by means of the sensors 44 and the parameters 46, are stored, and these are taken as a basis for creating a new calendar entry 34, at least when the training program or another activity that changes the state of health is carried out. The time at which the beneficiary will probably be in a situation that is the same as or at least similar to the current situation 50 is used for the new calendar entry 34. Therefore, when this time occurs, that is to say when the new calendar entry 34 occurs, it is likely that the beneficiary will also carry out the activity that changes the state of health. When the method 38 for operating the hearing device system 2 is carried out again, this calendar entry is then used as the applicable parameter 46 and therefore, at least implicitly, is used to determine whether the suitable time 54 is then at hand.

If, on the other hand, the beneficiary does not pursue the intervention, the touchscreen 36 is used in a fifth work step 58 to ask why the beneficiary does not pursue the intervention, and the applicable input is captured via the touchscreen 36.

In a subsequent sixth work step 60, the input and also the measurement data and parameters 46 denoting the current situation 50 that are captured by means of the sensors 44 are taken as a basis for creating a new calendar entry 34 or adapting an existing calendar entry 34. This involves considering why the beneficiary has not pursued the intervention. A time at which the beneficiary will probably be in a situation that is not the same as or similar to the current situation 50 is therefore chosen for the new calendar entry 34. This means that the beneficiary will not be excessively disrupted when they are in a situation similar to the current situation 50.

The invention is not limited to the exemplary embodiment described hereinabove. Rather, other variants of the invention can also be derived therefrom by a person skilled in the art without departing from the subject matter of the invention. In particular, all individual features described in connection with the exemplary embodiment can furthermore also be combined with one another in a different way without departing from the subject matter of the invention.

The following is a summary list of reference numerals and the corresponding structure used in the above description of the invention:
2 hearing device system
4 hearing device
6 housing
8 microphone
10 electronics
12 receiver
14 battery
16 acceleration sensor
18 communication apparatus
20 signal transfer connection
22 further communication apparatus
24 further device
26 further housing
28 control unit
30 GPS sensor
32 memory
34 calendar entry
36 touch screen
38 method for operating a hearing device system
40 method for operating a hearing device
42 first work step
44 sensor
46 parameter
48 second work step
50 current situation
52 third work step
54 suitable time
56 fourth work step
58 fifth work step
60 sixth work step

1. A method for operating a hearing device having a sensor, which comprises the steps of:
determining a current situation via the sensor; and
taking the current situation as a basis for checking whether there is a suitable time for an intervention for improving a state of health.

2. The method according to claim 1, which further comprising selecting the sensor from the group consisting of a microphone and an acceleration sensor.

3. The method according to claim 1, wherein the suitable time is at hand when there is an opportunity for an activity that changes the state of health.

4. The method according to claim 1, wherein the suitable time is at hand when no other persons are present.

5. The method according to claim 1, wherein the suitable time is at hand when a current reproduction setting does not correspond to the current situation.

6. A method for operating a hearing device system having a hearing device and a portable further device, which comprises the steps of:
operating the hearing device according to the sub-steps of:
determining a current situation via a sensor of the hearing device; and
taking the current situation as a basis for checking whether there is a suitable time for an intervention for improving a state of health;
providing, via the further device, a parameter that is further used for determining the current situation.

7. The method according to claim 6, which further comprises selecting the parameter from the group consisting of a calendar entry and a current position.

8. The method according to claim 6, wherein the suitable time is at hand when the hearing device is not connected to the further device for signal transfer purposes.

9. The method according to claim 6, wherein the suitable time is at hand when the hearing device is not worn on a body of a beneficiary.

10. A hearing device, comprising:
a sensor programmed to:
determine a current situation via said sensor; and
take the current situation as a basis for checking whether there is a suitable time for an intervention for improving a state of health.
